(19) United States
(12) Patent Application Publication
(10) Pub. No.: US 2023/0381248 A1
(43) Pub. Date: Nov. 30, 2023

(54) METHOD FOR TREATING AND/OR PREVENTING ATOPY AND ALLERGIC DISEASES BY USING BACTERIA OR COMPOSITIONS HAVING 7 ALPHA-DEHYDROXYLASE ACTIVITY, AND/OR BILE ACID RECEPTORS FXR AND/OR TGR5 AGONISTS

(71) Applicants: NATIONAL TAIWAN UNIVERSITY HOSPITAL, Taipei City (TW); National Taiwan University, Taipei City (TW)

(72) Inventor: Miao-Tzu HUANG, Taipei City (TW)

(21) Appl. No.: 18/135,394

(22) Filed: Apr. 17, 2023

Related U.S. Application Data
(60) Provisional application No. 63/346,291, filed on May 26, 2022.

(30) Foreign Application Priority Data
Nov. 30, 2022 (TW) 111146036

(51) Int. Cl. A61K 35/742; A61K 38/17; A61P 37/08; A61P 11/00

(52) U.S. Cl. CPC A61K 35/742; A61K 38/177; A61P 37/08; A61P 11/00

(57) ABSTRACT

The present disclosure provides a method for treating and/or preventing atopy and allergic diseases by using bacteria or compositions having 7 alpha-dehydroxylase activity, and the bile acid receptors FxR and/or TGR5 agonists. Bacteria or compositions having 7 alpha-dehydroxylase activity, and the bile acid receptors FxR and/or TGR5 agonists of the present disclosure can induce immune-regulatory leukocytes, and alleviate the severity of allergic airway diseases.

### CROSS-REFERENCE TO RELATED APPLICATION

This application claims priorities of Provisional Application No. 63/346,291, filed on May 26, 2022, and Taiwan patent application No. 111146036, filed on Nov. 30, 2022, the contents of which are incorporated herein in their entirety by reference.

### BACKGROUND OF THE INVENTION

### 1. Field of the Invention

The present invention relates to a method for treating and/or preventing atopy and allergic diseases by using bacteria or compositions having 7 alpha-dehydroxylase activity, and/or the bile acid receptors Farnesoid X receptor (FxR) and/or Takeda G protein-coupled receptor 5 (TGR5) agonists.

### 2. The Prior Art

Allergic diseases such as allergic rhinitis, atopic dermatitis, allergic airway diseases (e.g. allergic asthma) and food allergies have become more common in many countries. These diseases not only affect the quality of life of individuals, but also become social medical burden. Allergy is a T helper 2 cell (Th2 cell) response involving both T cells and B cells. Th2 responses are characterized by the production of certain cytokines, including interleukin (IL) IL-4, IL-5, IL-13, and the production of total immunoglobulin (Ig) E, antigen-specific IgE, and IgG1. Th2 cytokine production by Th2 cells enhances IgE production and eosinophil aggregation. Among Th2 cytokines, IL-4 and IL-5 are important for the differentiation, maturation and recruitment of eosinophils, while IL-13 directly enhances mucus hypersecretion and airway hyperresponsiveness (AHR). Th2 cytokines are potential targets for the treatment of asthma and other Th2-related diseases. The production of cytokines is a T-cell response while the production of immunoglobulins is considered a B-cell response. Th1 cells can suppress Th2 responses by secreting interferon (IFN)-γ, IgG2a, IL-2 and IL-3. Therefore, modulating the immune response by suppressing Th2 cell responses while enhancing Th1 responses is expected to be useful in the treatment of allergies and other Th2-dominated diseases. For example, asthma is a chronic complex respiratory disease caused by a variety of airway obstruction, airway eosinophilic inflammation and bronchial hyperresponsiveness. In addition to using the antagonistic effect of Th1 and Th2 to control allergic inflammatory reactions, the immune system also has a group of immune-regulatory leukocytes, such as myeloid-derived suppressor cells (MDSCs) and regulatory T-cell (Treg). The activation of these cells can effectively reduce the inflammatory response.

However, most of the pharmaceutical compositions or food compositions currently used to treat and/or prevent allergic diseases have side effects or poor effects. In order to solve the above-mentioned problems, those skilled in the art urgently need to develop a novel pharmaceutical composition or food composition for treating and/or preventing atopy and allergic diseases for the benefit of a large group of people in need thereof.

### SUMMARY OF THE INVENTION

A primary objective of the present invention is to provide a method for treating and/or preventing atopy and allergic disease, comprising administering to a subject in need thereof a composition comprising an effective amount of a bacterium, a gene vector, composite or extract, and/or bile acid receptors FxR and/or TGR5 agonists, wherein the bacterium, the gene vector, composite or extract has 7 alpha-dehydroxylase activity.

According to an embodiment of the present invention, the composition induces immune-regulatory leukocytes.

According to an embodiment of the present invention, the immune-regulatory leukocytes comprise a polymorphonuclear myeloid-derived suppressor cell (PMN-MDSC) and a regulatory T-cell (Treg).

According to an embodiment of the present invention, the allergic disease is allergic airway disease.

According to an embodiment of the present invention, the effective amount of the bile acid receptors FxR and/or TGR5 agonists is 24-36 mg/kg/day.

According to an embodiment of the present invention, the bacterium is Clostridium scindens.

According to an embodiment of the present invention, the composition is a pharmaceutical composition or a food composition.

According to an embodiment of the present invention, the composition further comprises a pharmaceutically acceptable carrier or an edible material.

According to an embodiment of the present invention, the pharmaceutical composition is in a dosage form for parenteral administration.

According to an embodiment of the present invention, the pharmaceutical composition is in a dosage form for oral administration.

In summary, the bacteria, the gene vector, composite or extract having 7 alpha-dehydroxylase activity, and/or the bile acid receptors FxR and/or TGR5 agonists have the effect on inducing immune-regulatory leukocytes (including polymorphonuclear myeloid-derived suppressor cell (PMN-MDSC) and regulatory T-cell (Treg)), and alleviating ovalbumin (OVA)-induced allergic airway disease.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the relative content of gut microbial profile in Clostridium scindens-voided (GN₁₂) and Clostridium scindens (CS) mice colonies, in which the number on the abscissa indicates the mouse number, NB represents 3-week-old (w/o) new-born CS mice around the time of weaning.

FIG. 2 depicts the protocol of the model of allergic airway disease, in which OVA represents ovalbumin.

FIGS. 3A-3C show the effects of Clostridium scindens in mitigating the severity of OVA-induced allergic airway disease, in which FIG. 3A shows seral OVA-specific IgE; FIG. 3B shows leukocyte subsets in bronchoalveolar lavage fluids (BALF) analyzed by flow cytometry using cell surface-specific markers; Mac represents macrophage, Eos represents eosinophil, Neu represents neutrophil, Lym represents lymphocyte; FIG. 3C shows interleukin (IL)-5 content in the BALF measured by enzyme-linked immunosorbent assay (ELISA). N=7 mice/group. **P<0.01; comparison was made by un-paired t-test.

FIGS. 4A-4C show the effects of Clostridium scindens in inducing both innate and adaptive immune-regulatory cells analyzed by flow cytometry, in which FIG. 4A shows that the bronchoalveolar lavage fluids and spleen of CS mice had more CD11b⁺Ly6G⁺Ly6C^{lo} polymorphonuclear myeloid-derived suppressor cells (PMN-MDSC) compared with Clostridium scindens-voided GN₁₂ mice (“+” represents expression of this marker, “lo” represents a low expression level of this marker); FIG. 4B shows that compared with Clostridium scindens-voided GN₁₂ mice, the bronchoalveolar lavage fluid (BALF) and spleen (Spl) of CS mice had more regulatory T-cell (Treg) population; FIG. 4C shows that the spleen cells of OVA-sensitized CS mice produced more regulatory T-cell than the spleen cells of OVA-sensitized GN₁₂ mice after being stimulated for 3 days in vitro. BALF represents bronchoalveolar lavage fluid; FoxP3 is an intracellular specific marker of regulatory T-cell and acts as a major regulator in the development and function of regulatory T-cell; N=7 mice/group. **P<0.01, ***P<0.001; comparison between groups was made by un-paired t-test.

FIG. 5 shows the difference in bile acid profiles between CS mice and Clostridium scindens-voided GN₁₂ mice. *P<0.05, **P<0.01, ***P<0.001; un-paired t-test.

FIG. 6 depicts the protocol for the intervention by FXR and TGR5 agonists in an allergic airway disease model.

FIGS. 7A and 7B show the effects of FXR and TGR5 signaling in inducing both innate and adaptive immune-regulatory cells (i.e., PMN-MDSC and Treg), in which FIG. 7A shows CD11b⁺Ly6G⁺Ly6C^{lo} PMN-MDSC in various tissue sites after aerosol OVA challenge analyzed by flow cytometry; FIG. 7B shows Treg population in various tissue sites after aerosol OVA challenge analyzed by flow cytometry. BALF represents bronchoalveolar lavage fluid, PB represents peripheral blood, BM represents bone marrow, Spl represents spleen. Ctrl represents control. FoxP3 is an intracellular specific marker of regulatory T-cell and acts as a major regulator in the development and function of regulatory T-cell. **P<0.01; ***P<0.001, comparison was made by analysis of variance (ANOVA).

FIGS. 8A-8D show the effects of FxR and TGR5 signaling in alleviating the severity of OVA-induced allergic airway disease, in which FIG. 8A shows seral OVA-specific IgE; FIG. 8B shows total and differential leukocyte counts in BALF analyzed by flow cytometry using cell surface-specific markers; Mac represents macrophage, Eos represents eosinophil, Neu represents neutrophil, Lym represents lymphocyte; FIG. 8C shows interleukin (IL)-5 content in the BALF; FIG. 8D shows that airway resistance in response to escalating concentrations of the bronchoconstrictor, methacholine, was measured as an indicator of airway hyper-responsiveness (AHR) at one day after the 3^{rd} aerosol OVA challenge. ***P<0.001, OVA-25 compared to Neg Ctrl-25 and FxR-25; ##P<0.01, OVA-25 compared to TGR5-25. Neg Ctrl represents healthy control. N=12-14 mice/group.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, the terms “a”, “the” and similar terms used in the present specification (especially in the claims of the patent application described later) should be understood to include both singular and plural forms.

The first composition provided according to the present invention can be a pharmaceutical composition or a food composition. The pharmaceutical composition provided according to the present invention can be used for treating and/or preventing atopy and allergic diseases. The food composition provided according to the present invention can also be used for treating and/or preventing atopy and allergic diseases.

The pharmaceutical composition provided according to the present invention can be a medicament, a nutritional supplement, a health food, an external supply, or any combination thereof, and can further comprise a pharmaceutically acceptable excipient, carrier, adjuvants, and/or food additives.

The pharmaceutical composition provided according to the present invention can be in any suitable form, without special limitations, and is in a corresponding appropriate dosage form depending on the intended use. For example, without limitation, the pharmaceutical composition can be administered to a subject in need thereof by oral or parenteral (e.g., intraperitoneal, but not limited to) administration.

According to the present invention, the pharmaceutical composition can be manufactured to a dosage form suitable for parenteral or oral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by an oral route selected from the group consisting of: chewable tablet, sublingual tablet, oral sprays, sterile powder, troche, lozenge, pill, capsule, dispersible powder or granule, solution, suspension, emulsion, syrup, elixir, slurry, and nasogastric tube.

The pharmaceutical composition according to the present invention may be administered by a parenteral route selected from the group consisting of: intraperitoneal injection, subcutaneous injection, intraepidermal injection, intradermal injection, intramuscular injection, intravenous injection, intralesional injection, sublingual administration, transdermal administration, and nasal sprays.

According to the present invention, the pharmaceutical composition may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. In addition, the external supply composition according to the present invention may further comprise an acceptable adjuvant that is widely used in external use manufacturing techniques. For example, the acceptable adjuvant may comprise one or more reagents selected from the group consisting of: solvent, gelling agent, active agent, preservative, antioxidant, screening agent, chelating agent, surfactant, coloring agent, thickening agent, filler, fragrance, and odor absorber. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), aqueous solution containing alcohol, and combinations thereof.

Taking the dosage form suitable for transdermal administration as an example, the pharmaceutical composition provided according to the present invention can be in the form of patches, lotions, creams, gels (for example hydrogels), pastes (for example: dispersion paste, ointment), spray, or solution (for example: suspension), etc., but not limited to this.

The pharmaceutical composition provided according to the present invention can be administered with different administration frequencies such as once a day, multiple times a day, or once a few days, depending on the individual's needs, age, weight and health status. In the pharmaceutical composition provided according to the present invention, the content ratio of the bacteria or compositions having 7 alpha-dehydroxylase activity, and the bile acid receptors FxR and/or TGR5 agonists in the composition can be adjusted according to actual application requirements. In addition, the pharmaceutical composition may additionally contain one or more other active ingredients (such as anti-allergy drug) as needed, or be used in combination with drugs containing the one or more other active ingredients in order to further strengthen the effect of the pharmaceutical composition or increase the flexibility and deployment degree of the formulation, as long as the other active ingredients have no adverse effect on the benefits of the active ingredients of the present invention (i.e., the bacteria or compositions having 7 alpha-dehydroxylase activity, and the bile acid receptors FxR and/or TGR5 agonists).

Optionally, the pharmaceutical composition or food composition provided according to the present invention may additionally contain an appropriate amount of additives, such as flavoring agents, toners, colorants, etc. that can improve the taste and visual perception of the pharmaceutical composition or food composition when taking it, and buffers, preservatives, antibacterial agents, antifungal agents, etc. that can improve the stability and storage of the pharmaceutical composition or food composition.

The food composition provided by the present invention can be a food product, and is formulated with edible materials to include but not limited to: beverages, fermented foods, bakery products, health foods, nutritional supplements, and dietary supplements.

According to the present invention, the edible material is selected from the group consisting of: water, fluid milk products, milk, concentrated milk; fermented milk such as yogurt, sour milk, frozen yogurt, and lactic acid bacteria-fermented beverages; milk powder; ice cream; cream cheeses; dry cheeses; soybean milk; fermented soybean milk; vegetable-fruit juices; juices; sports drinks; confectionery; jellies; candies; infant formulas; health foods; animal feeds; Chinese herbals; and dietary supplements.

According to the present invention, the food product can be regarded as a food additive, which is added during the preparation of raw materials by conventional methods, or added in the production process of food, and formulated with any edible material into food products for human and non-human animals to eat.

The beverage, fermented food, bakery product, health food, nutritional supplements, and dietary supplements provided according to the present invention can be eaten with different frequencies such as once a day, multiple times a day, or once a few days, depending on the individual's age, weight and health status. The content of the bacteria or compositions having 7 alpha-dehydroxylase activity, and the bile acid receptors FxR and/or TGR5 agonists in the beverage, fermented food, bakery product, health food, nutritional supplement and dietary supplement provided according to the present invention can also be adjusted according to the needs of specific groups, for example, adjusted to be taken every day amount.

For beverages, fermented foods, bakery products, health foods, nutritional supplements and/or dietary supplements provided according to the present invention, the recommended usage amount can be marked on their outer packaging, the usage standards and conditions for specific groups (for example insomniacs, depression patients, pregnant woman, etc.), or recommendations for co-administration with other foods or medicines, etc., to facilitate users to take them without the guidance of doctors, pharmacists or relevant deacons without safety concerns. In the food composition provided according to the present invention, the aspects of the bacteria or compositions having 7 alpha-dehydroxylase activity, and the bile acid receptors FxR and/or TGR5 agonists and related applications are as described above.

### Example 1

Relative Content of Gut Microbial Profile in Clostridium scindens-Voided (GN₁₂) and Clostridium scindens (CS) Mice Colonies

To investigate the immune regulatory effect and the role of Clostridium scindens (ATCC 35704) in prevention and treatment of allergic diseases, we established GN₁₂ mice with 12 strains of representative specific intestinal microorganisms (see Narushima, S., Itoh, K., Takamine, F. & Uchida, K. Absence of cecal secondary bile acids in gnotobiotic mice associated with two human intestinal bacteria with the ability to dehydroxylate bile acids in vitro. Microbiology and Immunology 43, 893-897 (1999).; Studer, N. et al. Functional intestinal bile acid 7α-dehydroxylation by Clostridium scindens associated with protection from Clostridium difficile infection in a gnotobiotic mouse model. Frontiers in Cellular and Infection Microbiology 6 (2016).; Eberl, C. et al. Reproducible colonization of germ-free mice with the oligo-mouse-microbiota in different animal facilities. Frontiers in Microbiology 10 (2020).) by colonizing germ-free BALB/c mice provided by National Laboratory Animal Center with two oral gavages of a simplified microbial consortium containing 12 species of bacteria representative of the major murine intestinal microbial phyla but lacking 7α-dehydroxylase activity. In parallel, mice colonized with Clostridium scindens (CS) were established by colonizing the GN₁₂ mice with two oral gavages of Clostridium scindens (containing 7α-dehydroxylase). Colonization status of the GN₁₂ and the CS mice was monitored by quantitative polymerase chain reaction (qPCR) using microbe-specific primer pairs (see Brugiroux, S. et al. Genome-guided design of a defined mouse microbiota that confers colonization resistance against Salmonella enterica serovar Typhimurium. Nature Microbiology 2, 16215 (2016).)

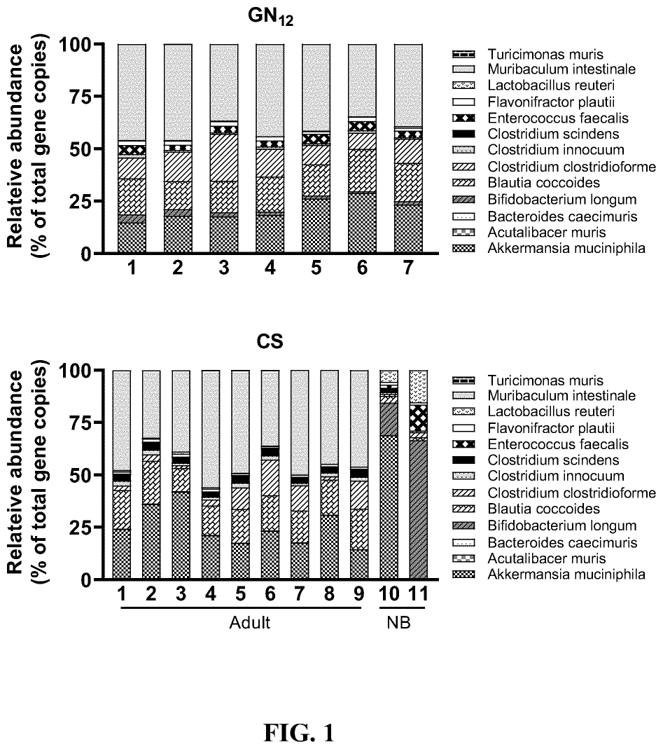

The relative content of gut microbial profile in Clostridium scindens-voided (GN₁₂) and Clostridium scindens (CS) mice colonies is shown in FIG. 1, in which the number on the abscissa indicates the mouse number, NB represents 3-week-old (w/o) new-born CS mice around the time of weaning.

### Example 2

### Establishment of Model of Allergic Airway Disease

The model uses ovalbumin (OVA) as the allergen to induce allergic airway inflammation of the mice. Briefly, allergen sensitization was induced in 5 w/o BALB/c mice by two intraperitoneal injection (i.p.) of 50 μg ovalbumin (OVA) plus 2 mg alum at 10-14-day interval. One week after the second OVA i.p. sensitization, 30-min aerosol 5% OVA inhalation challenge (ih) was performed for 3 consecutive days.

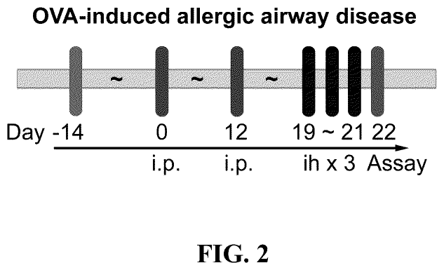

The protocol of the model of allergic airway disease is shown in FIG. 2.

### Example 3

Effects of Clostridium scindens in Mitigating the Severity of OVA-Induced Allergic Airway Disease

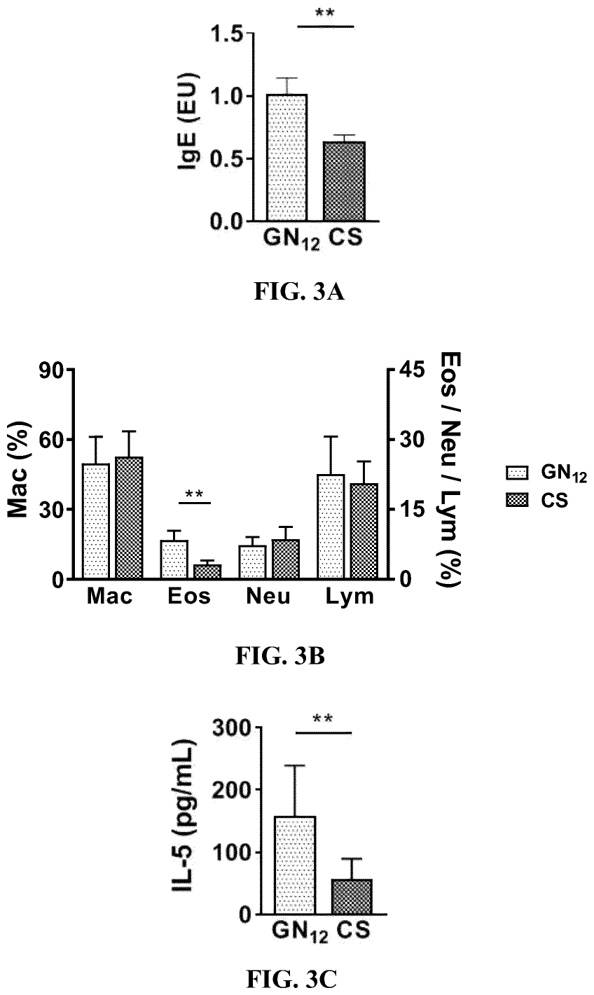

The effects of Clostridium scindens in mitigating the severity of OVA-induced allergic airway disease are investigated in this example. Allergic airway disease was induced in 5 w/o GN₁₂ and CS mice as depicted in FIG. 2. One day after the 3^{rd} aerosol OVA inhalation challenge, sera and bronchoalveolar lavage fluids (BALF) were collected and parameters reflecting the severity of allergic inflammation were tested. The results are shown in FIGS. 3A-3C, in which FIG. 3A shows seral OVA-specific IgE; FIG. 3B shows leukocyte subsets in bronchoalveolar lavage fluids (BALF) analyzed by flow cytometry using cell surface-specific markers; Mac represents macrophage, Eos represents eosinophil, Neu represents neutrophil, Lym represents lymphocyte; FIG. 3C shows interleukin (IL)-5 content in the BALF measured by enzyme-linked immunosorbent assay (ELISA). N=7 mice/group. **P<0.01; comparison was made by un-paired t-test.

It can be concluded from FIGS. 1-3C that Clostridium scindens colonization is effective in mitigating severity of allergic airway disease.

### Example 4

Effects of Clostridium scindens in Inducing Both Innate and Adaptive Immune-Regulatory Cells

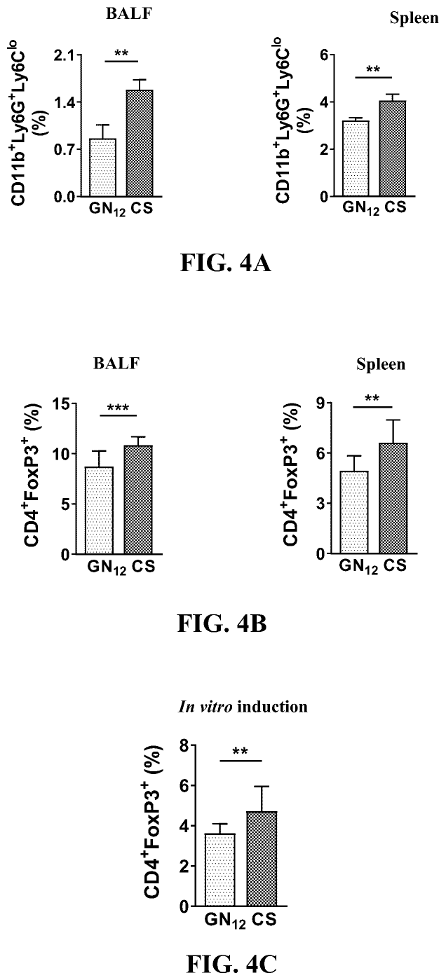

The effects of Clostridium scindens in inducing both innate and adaptive immune-regulatory cells are investigated in this example. As shown in FIG. 2, OVA was used to sensitize BALB/c mice and induce asthmatic responses. Samples were collected one day after the 3^{rd} aerosol OVA inhalation challenge and analyzed by flow cytometry. The results are shown in FIGS. 4A-4C, in which FIG. 4A shows that the bronchoalveolar lavage fluids and spleen of CS mice had more CD11b⁺Ly6G⁺Ly6C^{lo} polymorphonuclear myeloid-derived suppressor cells (PMN-MDSC) compared with Clostridium scindens-voided GN₁₂ mice (“+” represents expression of this marker, “lo” represents a low expression level of this marker); FIG. 4B shows that compared with Clostridium scindens-voided GN₁₂ mice, the bronchoalveolar lavage fluid (BALF) and spleen (Spl) of CS mice had more regulatory T-cell (Treg) population; FIG. 4C shows that the spleen cells of OVA-sensitized CS mice produced more regulatory T-cell than the spleen cells of OVA-sensitized GN₁₂ mice after being stimulated with OVA (200 μg/ml) for 3 days in vitro. BALF represents bronchoalveolar lavage fluid; FoxP3 is an intracellular specific marker of regulatory T-cell and acts as a major regulator in the development and function of regulatory T-cell; N=7 mice/group. **P<0.01, ***P<0.001; comparison between groups was made by un-paired t-test.

This example shows the effects of Clostridium scindens in inducing immune-regulatory cells, including polymorphonuclear myeloid-derived suppressor cells (PMN-MDSC) and regulatory T cells (Treg).

### Example 5

Bile Acid Profiles Between CS Mice and Clostridium scindens-Voided GN₁₂ Mice

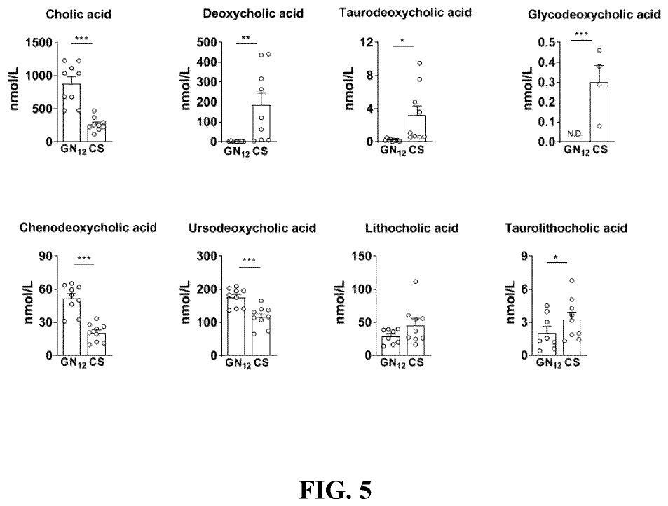

The difference in bile acid profiles between CS mice and Clostridium scindens-voided GN₁₂ mice was investigated in this example. Stool pellets were collected at 8 w/o from both groups of mice. Metabolites in stool pellets were extracted by using acetonitrile-methanol extract solvent containing 1% formic acid and bile acid composition was analyzed by mass spectrometry (Agilent 1290 Infinity series UHPLC-PRM-MS System, Agilent Technologies). The results are shown in FIG. 5. *P<0.05, **P<0.01, ***P<0.001; un-paired t-test.

This example shows that mice colonized with Clostridium scindens (CS) exhibited a bile acid profile enriched with secondary bile acids, and Clostridium scindens-voided GN₁₂ mice were unable to produce secondary bile acids. Secondary bile acids are deoxycholic acid, taurodeoxycholic acid, glycodeoxycholic acid, lithocholic acid and taurolithocholic acid, verifying the metabolic effect of the 7 alpha-dehydroxylase exclusively expressed by Clostridium scindens.

According to this embodiment, any microorganisms, gene vectors, composites, or extracts that exhibit 7 alpha-dehydroxylase activity, which can carry out primary to secondary bile acid biotransformation, are all applicable to the uses of this application.

### Example 6

### Protocol for Intervention by FXR and TGR5 Agonists in Allergic Airway Disease Model

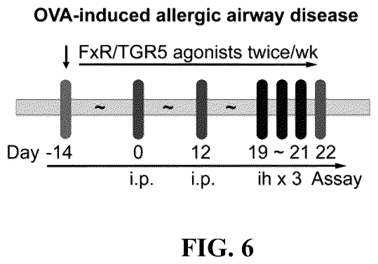

This example uses ovalbumin (OVA) as the allergen as in Example 2 to induce the allergic airway disease model of allergic airway inflammation in the mice. OVA-induced allergic airway disease was induced in 5 w/o BALB/c mice according to the protocol as depicted. To investigate the effects of the FxR and TGR5 bile acid receptor signaling in allergic airway disease, mice were given oral gavage of either FxR or TGR5 agonist (i.e., INT-747 (6-ECDCA) or INT-777 (6-EMCA)) 30 mg/kg/day twice a week since 3 w/o, i.e. starting 14 days before i.p. OVA sensitization. Age- and sex-matched control mice were given the vehicle containing methyl cellulose. The protocol for the intervention by FXR and TGR5 agonists in an allergic airway disease model is shown in FIG. 6.

### Example 7

### Effects of FXR and TGR5 Signaling in Inducing Both Innate and Adaptive Immune-Regulatory Cells

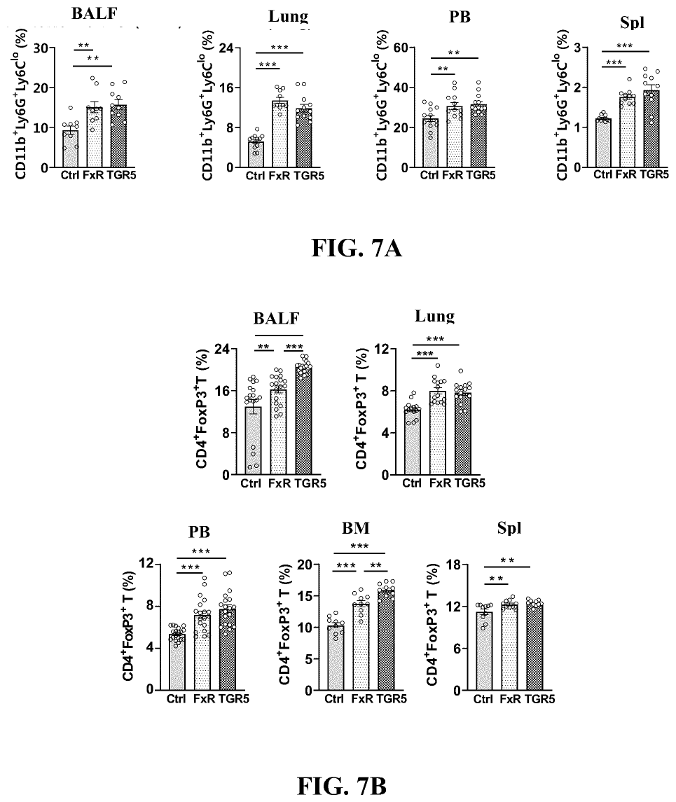

The effects of FXR and TGR5 signaling in inducing both innate and adaptive immune-regulatory cells (i.e., PMN-MDSC and Treg) are investigated in this example. Mice were pre-treated by FxR and TGR5 agonists, followed by allergen sensitization and induction of allergic airway disease as depicted in FIG. 6. The results are shown in FIGS. 7A and 7B, in which FIG. 7A shows CD11b⁺Ly6G⁺Ly6C^{lo} polymorphonuclear myeloid-derived suppressor cells (PMN-MDSC) in various tissue sites after aerosol OVA challenge analyzed by flow cytometry; FIG. 7B shows that regulatory T-cells (Treg) population in various tissue sites after aerosol OVA challenge analyzed by flow cytometry. BALF represents bronchoalveolar lavage fluid, PB represents peripheral blood, BM represents bone marrow, Spl represents spleen. Ctrl represents control. FoxP3 is an intracellular specific marker of regulatory T-cell and acts as a major regulator in the development and function of regulatory T-cell. **P<0.01; ***P<0.001, comparison was made by analysis of variance (ANOVA).

These data indicate that bile acid signaling derived from either FxR or TGR5 can induce the expansion of the innate immune-regulatory cells, PMN-MDSC and the adaptive immune-regulatory cell, Treg. This example shows that FXR and TGR5 signaling has the effect on inducing immune-regulatory leukocytes, including PMN-MDSC and Treg.

### Example 8

### Effects of FxR and TGR5 Signaling in Alleviating Severity of OVA-Induced Allergic Airway Disease

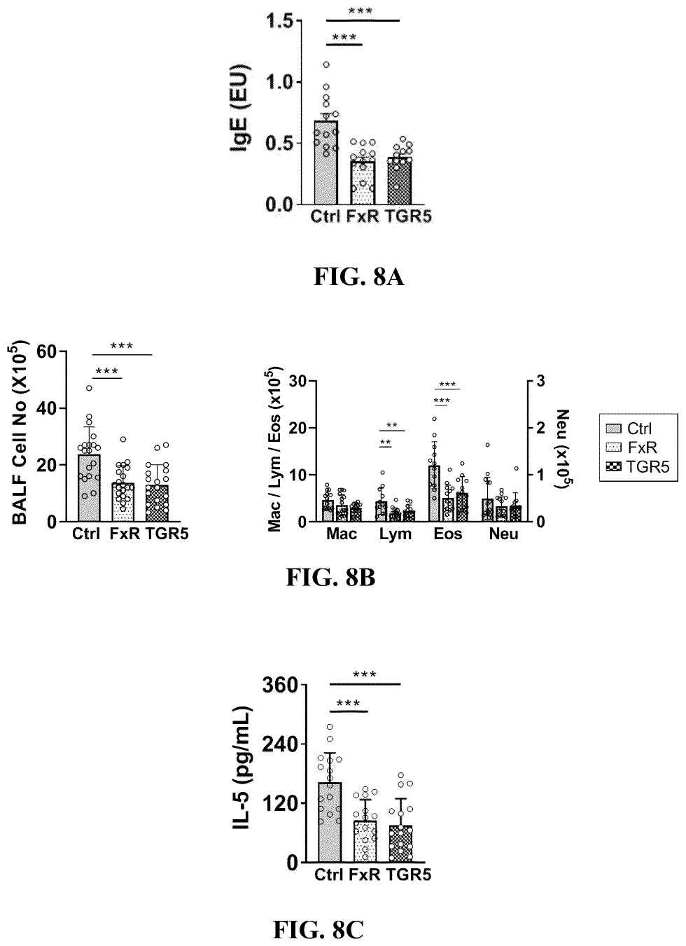

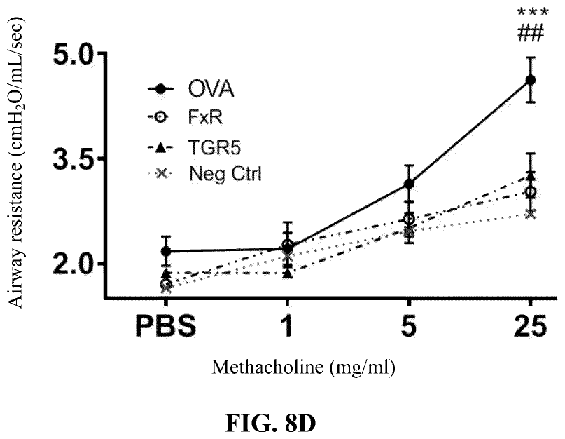

The effects of FxR and TGR5 signaling in alleviating the severity of OVA-induced allergic airway disease are investigated in this example Mice were pre-treated by FxR and TGR5 agonists, followed by sensitization and asthmatic response of mice as depicted in FIG. 6. One day after the 3^{rd} aerosol OVA challenge, sera and bronchoalveolar lavage fluids (BALF) were collected and parameters reflecting the severity of allergic inflammation were tested. The results are shown in FIGS. 8A-8D, in which FIG. 8A shows seral OVA-specific IgE; FIG. 8B shows total and differential leukocyte counts in BALF analyzed by flow cytometry using cell surface-specific markers; Mac represents macrophage, Eos represents eosinophil, Neu represents neutrophil, Lym represents lymphocyte; FIG. 8C shows interleukin (IL)-5 content in the BALF; FIG. 8D shows that airway resistance in response to escalating concentrations of the bronchoconstrictor, methacholine, was measured as an indicator of airway hyper-responsiveness (AHR) at one day after the 3^{rd} aerosol OVA challenge. ***P<0.001, OVA-25 compared to Neg Ctrl-25 and FxR-25; ##P<0.01, OVA-25 compared to TGR5-25. Neg Ctrl represents healthy control. N=12-14 mice/group.

This embodiment shown here demonstrated that the mice receiving FxR and TGR5 agonist pretreatment developed significantly milder airway disease than control mice receiving vehicle, and the asthmatic phenotype can be reverted to a state close to that of the healthy mice. All the four parameters indicative of allergic airway disease severity in FIGS. 8A-8D were mitigated in the FxR- and TGR5-treated mice. These results support a protective role of FxR and TGR5 in allergic airway disease.

The above results demonstrated that the activation of either one of FxR or TGR5 can improve the severity of allergic airway diseases. Without causing general immune suppression of conventional pharmaceutical regiments, the present invention uncovers the induction of immune-regulatory cells by Clostridium scindens and via activation of FxR and TGR5. In particular, the FxR and TGR5 signaling has the effect on alleviating the severity of OVA-induced allergic airway disease.

In summary, the bacteria, the gene vector, composite or extract having 7 alpha-dehydroxylase activity, and/or the bile acid receptors FxR and/or TGR5 agonists have the effect on inducing immune-regulatory leukocytes (including polymorphonuclear myeloid-derived suppressor cell (PMN-MDSC) and regulatory T-cell (Treg)), and alleviating allergen (e.g., ovalbumin (OVA))-induced allergic airway disease.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating and/or preventing atopy and allergic disease, comprising administering to a subject in need thereof a composition comprising an effective amount of a bacterium, a gene vector, composite or extract, and/or bile acid receptors FxR and/or TGR5 agonists, wherein the bacterium, the gene vector, composite or extract has 7 alpha-dehydroxylase activity.

2. The method according to claim 1, wherein the composition induces immune-regulatory leukocytes.

3. The method according to claim 2, wherein the immune-regulatory leukocytes comprise a polymorphonuclear myeloid-derived suppressor cell (PMN-MDSC) and a regulatory T-cell (Treg).

4. The method according to claim 1, wherein the allergic disease is allergic airway disease.

5. The method according to claim 1, wherein the effective amount of the bile acid receptors FxR and/or TGR5 agonists is 24-36 mg/kg/day.

6. The method according to claim 1, wherein the bacterium is Clostridium scindens.

7. The method according to claim 1, wherein the composition is a pharmaceutical composition or a food composition.

8. The method according to claim 1, wherein the composition further comprises a pharmaceutically acceptable carrier or an edible material.

9. The method according to claim 7, wherein the pharmaceutical composition is in a dosage form for parenteral administration.

10. The method according to claim 7, wherein the pharmaceutical composition is in a dosage form for oral administration.
